(19) United States
(12) Patent Application Publication
(10) Pub. No.: US 2003/0157183 A1
(43) Pub. Date: Aug. 21, 2003

(54) Method for encapsulating fine solid particles in the form of microcapsules

(76) Inventor: Michel Perrut, Nancy (FR)

Correspondence Address: ALLEN BLOOM, C/O DECHERT, PRINCETON PIKE CORPORATION CENTER, P.O. BOX 5218, PRINCETON, NJ, 08543-5218, US

(21) Appl. No.: 10/333,038

(22) PCT Filed: Jul. 19, 2001
(86) PCT No.: PCT/FR01/02350

(30) Foreign Application Priority Data
Jul. 19, 2000 (FR) 0/09473

(51) Int. Cl. A61K 9/16; A61K 9/50; B01J 13/02; B01J 13/04

(52) U.S. Cl. 424/490; 264/4.1

(57) ABSTRACT

The invention concerns a method for encapsulating fine solid particles in the form of microcapsules. Said method is characterised in that it comprises steps which consist in forming a suspension of said particles in a liquid consisting of at least a coating agent in intimate contact with a fluid having a pressure lower than its critical pressure, so as to saturate said suspension, spraying said fluid-saturated suspension through expansion means, and collecting the microcapsules in the gas stream resulting from decompression.

The present invention relates to a method for encapsulating, in the form of microcapsules, fine solid particles such as in particular proteins. It also concerns the microcapsules obtained in accordance with such a method.

Numerous industries are known to employ solids in pulverulent form of which certain are in the form of complex particles, or capsules, comprising a “core” made of a certain matter, or active agent, and a coating or “peel” of another matter. This type of capsules, also called microcapsules when their diameter is smaller than about 100 μm, is used when the active agent is to be protected from the environment during conservation and/or employment thereof. Such microcapsules are used for example in reproduction graphics inks, in numerous cosmetic and dermatological preparations, and in certain pharmaceutical products.

In effect, the pharmaceutical industry, but also the cosmetic industry, requires novel galenic forms in order to improve the service rendered by certain molecules of therapeutical or dermatological interest. In particular, it is seeking the means for producing an efficient protection of certain molecules which would be destroyed as soon as they are absorbed by the digestive enzymes, or which would not be stable at conservation in the presence of oxygen or of the humidity of the air, or of light. Microcapsules respond well to this need.

Similarly, it is sometimes interesting to obtain a slow dissolution within the tissues or biological fluids such as blood or lymph. To that end, it is necessary to disperse the particles of active agent within a suitable coating allowing an appropriate diffusion of this active agent at the desired spot. In that case, another type of microcapsules will preferably be used, with so-called matricial structure, sometimes called microspheres to distinguish them from the preceding type, constituted by a mixture which is as homogeneous as possible of the particles of active agent within the coating agent; ideally, the active agent is literally dissolved within the coating agent.

The interest of these structures is such that numerous methods of obtaining have been described and, for certain, are exploited industrially. However, it will be noted that it is particularly difficult to manufacture such microspheres or microcapsules whose core is constituted by an active agent formed by proteins of therapeutical interest within an excipient, by reason of the great fragility of these proteins.

In effect, it is known that the denaturation of proteins is irreversible when they are taken to too high a temperature, or when they are placed in contact either with an organic medium, or with an aqueous medium of which the pH is outside their zone of stability, or with certain fluids taken to high pressure such as carbon dioxide. In effect, it is known that, unlike the conventional chemical or biological products, proteins are complex and fragile edifices whose biological activity is closely connected with their three-dimensional conformation which may be easily affected by the environment of the molecule, which has the effect of bringing about a generally irreversible destruction of its biological activity. Such a fragility is particularly great for numerous proteins of high therapeutical interest of which production is beginning to be carried out in accordance with new biotechnologies, but whose therapeutical implementation proves to be extremely delicate.

It is precisely an object of the present invention to describe a method for obtaining microcapsules with core-peel structure or with matricial structure otherwise called microspheres, and more particularly microcapsules allowing in particular proteins to be included within an excipient.

Furthermore, it is known, from numerous scientific publications and Patents, that it is possible to obtain microspheres or micro-particles which are complex and very fine (with a granulometry generally included between 1 and 10 μm, and nano-particles with a granulometry generally included between 0.1 and 1 μm) constituted by mixtures of different morphologies of an active agent and of an excipient, using methods employing supercritical fluids such as in particular carbon dioxide. The so-called “RESS” method is for example known, consisting in expanding very rapidly at low pressure a solution of a product to be atomized in a supercritical fluid, or the anti-solvent method called either SESS, or SEDS, or PCA, or ASES, which consists in spraying a solution of a product to be atomized in an organic or aqueous solvent within a stream of fluid in supercritical state. These methods make it possible to obtain a powder formed by very fine particles dispersed within a gaseous stream at low pressure (RESS method) or at high pressure (SAS method).

By reason of its very principle, the anti-solvent method inescapably involves the use of at least one organic solvent or co-solvent. This results in a certain number of drawbacks and in particular considerable problems of recovery of the solvent and of purification of the microcapsules obtained. This method also present a major drawback, namely that of not being able to be used for effecting encapsulation of fragile bio-molecules, such as in particular proteins, insofar as the majority of them are irreversibly denatured upon contact with an organic solvent.

Another method, described in Patent FR-2 753 639, consists in effecting coacervation of the coating agent initially dissolved in an organic solvent within which the particles to be coated are maintained in dispersion, said coacervation being provoked by an anti-solvent effect caused by the dissolution of a supercritical fluid or a liquefied gas in said organic solvent. The recovery of the capsules obtained is effected after complete extraction of the organic solvent by a stream of supercritical fluid or of liquefied gas, then decompression of the recipient in which the encapulsation was effected. This method therefore likewise presents the drawback of necessitating employment of an organic solvent within which the particles of active agent will be dispersed.

European Patent EP-A-0 744 99 discloses a method developed in accordance with a concept called PGSS (“Particle from gas-saturated solutions”) and which consists in dissolving a compressible fluid in a substance to be sprayed until a solution saturated with fluid is formed, then in decompressing this solution so that, on the one hand, it is sprayed in fine droplets, and, on the other hand, the resulting cooling of this decompression induces a solidification of the initial substance in the form of fine solid particles. This method also describes the production of micro-spheres constituted by a homogeneous mixture of two or more compounds which are initially mixed in the form of a homogeneous solution. It will be understood that such a method cannot be used for the micro-encapsulation of products such as proteins insofar as it imposes that these proteins mixed with their stabilization agents be placed in solution within the liquefied excipient.

Very similarly, Patent Application WO-98/15348 describes the application of the preceding concept to the manufacture of microcapsules constituted by particles of an active agent encapulsated in a polymer, by using a supercritical fluid which, on dissolving in the polymer, liquefies it to a temperature lower than the melting temperature of the polymer and allows the placing in suspension of the particles of the active agent within this liquid phase itself saturated with supercritical fluid, which suspension is then expanded to atmospheric pressure with the formation of microcapsules due to the solidification of the polymer around the particles of active agent.

A method of producing paint in powder form is also known from U.S. Pat. No. 5,399,597, in which there is made in a first, mechanically stirred recipient, a mixture comprising a polymer, a cross-linking agent and possibly other components coming within the usual composition of a paint (pigments, fillers) with carbon dioxide in the supercritical state, this mixture being taken to adequate temperature and pressure in order to obtain, after partial or total expansion of this mixture in a second recipient maintained at a pressure clearly lower than that of the first, a solid powder constituted by an intimate mixture of the different initial solid constituents. These particles therefore have a structure close to that of the micro-spheres defined hereinabove. It will be noted that this method employs carbon dioxide at supercritical pressure and that the temperature of use of the mixture in the first recipient is generally close to the temperature of melting or of vitreous transition of the polymer. It will be readily understood that such a method cannot be used for effecting encapsulation of fragile particles such as in particular proteins.

The present invention has for its object to propose a method for elaborating microcapsules constituted by particles of an active agent with a diameter generally smaller than 20 μm, and often smaller than 10 μm, dispersed within a coating agent, these microcapsules having an adjustable mean diameter which is preferably included between a minimum value equal to 2 to 3 times the mean diameter of the encapsulated particles and a maximum value equal to about 200 μm, and making it possible in particular to include proteins within an excipient.

The present invention thus has for its object a method for encapsulating fine solid particles in the form of microcapsules, characterized in that it comprises the steps consisting in:

forming, in an enclosure, a suspension of these particles in a liquid constituted by at least one coating agent in intimate contact with a fluid at a pressure lower than its critical pressure so as to provoke saturation of said suspension with this fluid,

spraying the fluid-saturated suspension through expansion means, and

collecting the microcapsules in the gas stream resulting from decompression.

The present invention is particularly intended for encapsulating very fine solid particles whose size is smaller than 20 μm and most often smaller than 10 μm with a view to obtaining microcapsules intended for preparations for therapeutical use in human or veterinary pharmacy, or in cosmetics or plant protection.

In a variant embodiment of the invention during the first step, an aqueous or organic solvent may be added to the coating agent. In such a form of embodiment, the gaseous fluid may entrain the solvent and the total elimination thereof may be obtained by means of a “stripping” of the microcapsules by a fluid taken to a supercritical pressure.

According to the invention, a continuous feed may be ensured of the fluid at subcritical pressure and of the suspension of the active agent in the coating agent inside an enclosure as well as a continuous drawing off of the products contained in this enclosure which are admitted in a spray nozzle disposed in a collector recipient maintained at a pressure close to atmospheric pressure.

In particular, a lipid or a mixture of lipids may be used as coating agent.

Furthermore, the particles may be placed in suspension at a temperature of the order of 50° C. to 60° C. and at a pressure of the order of 5 Mpa to 6 Mpa and the pressure during the collection will be close to atmospheric pressure.

The present invention also has for its object the microcapsules obtained in accordance with the method of the invention. The particles may be constituted by an active agent of alimentary, pharmaceutical, cosmetic, agrochemical or veterinary interest. These particles may in particular by constituted by a protein associated, or not, with a stabilization agent.

This invention is interesting in that it makes it possible to use a broad range of coating agents and not only polymers, and, even more surprizingly, that the fluid may be dissolved in large quantity in the suspension of active agent dispersed in the coating agent at a pressure significantly lower than its critical pressure.

This results in that, even under these subcritical pressure conditions, the melting temperatures of the polymers and of other compounds in which the fluid is soluble in noteworthy manner like the majority of lipids, will be significantly lowered, thus allowing the method according to the invention to be carried out at temperatures lower than the temperatures of degradation of the majority of the active agents. This invention is particularly advantageous when it is desired to encapsulate proteins in an excipient.

Moreover, unlike what was described in Patent EP-A-0 744 992, it is possible to spray a non-homogeneous mixture constituted by particles in suspension and not only a homogeneous solution, and this, even when the pressure of the fluid is lower than its critical pressure. As a result, the method according to the invention allows microcapsules to be obtained under conditions which are easier and less expensive to carry out in a large number of applications employing a wider variety of active agents and of coating agents.

These advantages are particularly interesting concerning the encapsulation of proteins which are in the form of a powder, generally obtained by lyophilization of a solution of proteins in the presence of stabilization agents.

These proteins, mixed with these stabilization agents, cannot, in fact, be placed in solution within the liquefied excipient, as required by the method described in afore-mentioned Patent EP-A-0 744 992. Moreover, the method according to the invention, apart from the fact that it allows a very large range of excipients, and not only a polymer, to be used, makes it possible to place the protein in contact with a fluid at pressure clearly lower than that employed according to the method described in afore-mentioned Patent Application WO-98/15348, thus considerably reducing the risk of denaturation of the protein, especially when the fluid used is carbon dioxide.

Various forms of embodiment of the present invention will be described hereinafter by way of non-limiting examples, with reference to the accompanying drawing, in which:

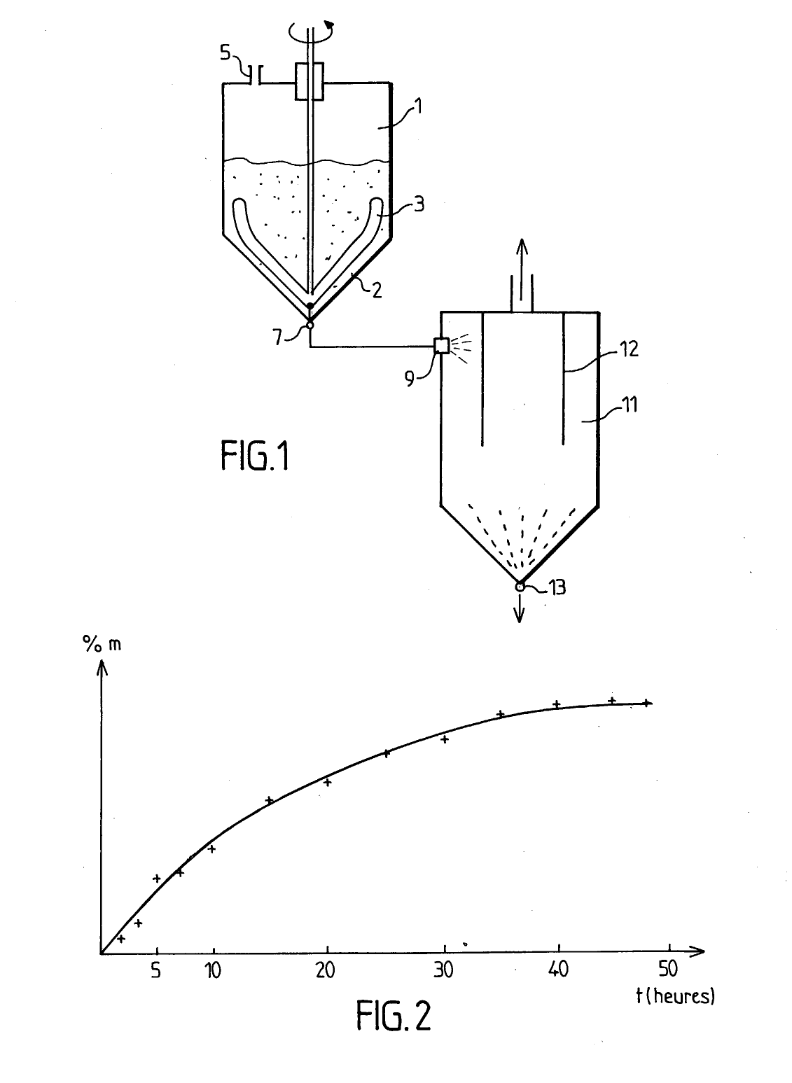

FIG. 1 is a schematic view representing the principle of implementation of the method according to the invention.

FIG. 2 is a curve showing the kinetics of salting-out of an active agent contained in a coating agent when the microcapsule is placed in water. It represents, as a function of time, the percentage of active agent which has dissolved in the water with respect to its quantity present in the particles.

FIG. 1 shows a device for carrying out the method according to the invention. This device comprises an enclosure 1 whose bottom 2 is conical and inside which a stirrer 3 is mounted for rotation about a vertical axis. The enclosure 1 is provided with an inlet 5 and a lower axial outlet 7 which is in communication with a spray nozzle 9 disposed in the upper part of a collector recipient 11. This collector recipient will preferably be of cyclonic type and will, to that end, comprise a cylindrical tube 12 creating an upper annular zone in which the gaseous flow laden with particles will be admitted in tangential manner. The collector recipient 11 comprises an extraction orifice 13 disposed in the bottom thereof through which the microspheres produced will be extracted.

The method according to the invention is generally carried out as described hereinbelow.

The particles of active agent as well as the coating agent are admitted via the admission orifice 5 into the enclosure 1, then a fluid at subcritical pressure is then introduced in the enclosure 1 at the desired temperature and pressure and the stirrer 3 is activated so as to produce a suspension of the particles of active agent within the fluid-saturated coating agent. After a lapse of time sufficient for equilibrium to be attained, the enclosure 1 contains a light phase constituted essentially by the fluid at subcritical pressure within which the coating agent and the active agent are virtually insoluble, and a heterogeneous heavy phase which is constituted by a suspension of the particles of active agent within the fluid-saturated coating agent.

This heavy phase is extracted via the orifice 7 and it is admitted, through the spray nozzle 9, into the collector recipient 11, which is maintained at a pressure close to atmospheric pressure, which subjects this heavy phase to a sudden decompression which makes it possible to spray it in fine droplets which are considerably cooled by the degassing of the fluid returned to low pressure, so that they solidify in the form of microcapsules.

Such an implementation may advantageously be conducted periodically.

When the coating agent can be placed in liquid phase at a temperature lower than the temperature of degradation of the active agent, it is possible, in another particularly interesting form of embodiment of the invention, to ensure the suspension of the active agent in a coating agent which was previously melted. The suspension thus produced and the fluid at subcritical pressure are introduced continuously in the enclosure 1 and the heavy phase obtained is drawn off to continuously conduct it within the enclosure 1, while maintaining constant the quantity of heavy phase present therein.

In an interesting variant of the invention, a small quantity of organic solvent is used for placing in liquid phase the coating agent at a temperature lower than its melting temperature, or at atmospheric pressure, or when it is saturated with fluid at subcritical pressure.

Such a form of embodiment is particularly interesting when the active agent is very sensitive to temperature, which is particularly the case of proteins. In pharmaceutical or cosmetic applications, preferably very volatile, non- or hardly toxic organic solvents will preferentially used, such as ethanol, n-propanol, isopropanol, acetone or ethyl acetate. In other applications, other alcohols may also be used, such as methanol, ketones or esters, or light hydrocarbons or halocarbons having between 3 and 8 carbon atoms.

It has been verified that the microcapsules thus obtained contained very low contents of residual solvent, particularly for the reason that the gaseous phase resulting from the decompression of the heavy phase entrains virtually all of this solvent, and this all the more so as it presents a greater volatile nature.

In the cases where, for certain specific applications, this organic solvent content must be reduced to very low levels, the residual solvent may be extracted without particular difficulty by subjecting the microcapsules to a brief treatment (so-called “stripping” extraction) by a stream of fluid taken to supercritical pressure, but taking care to maintain the temperature of this fluid below the melting temperature of the coating agent at the pressure in question.

Examples of implementation are presented hereinafter in order to illustrate in non-limiting manner the method according to the invention.

In all cases, the same equipment for encapsulation of particles and for collecting the microcapsules, which is schematically shown in FIG. 1, has been used.

The equipment used for carrying out the examples presented hereinafter is of semi-industrial size. It used carbon dioxide as fluid at subcritical pressure, with a service pressure of 8 MPa and a temperature range of from 0° C. to 150° C. The enclosure 1 under pressure had a total volume of 4 litres, and was provided with a stirrer 3 of anchor type moved by an electric motor at speed varying between 100 and 800 revs per minute. This enclosure 1 was constituted by a recipient terminated by a conical bottom with an angle of 45° and a diameter of 0.10 m, with a double envelope through which passes a heat-exchange fluid making it possible to maintain the temperature of the whole to the desired value. The collector recipient 11 was constituted by a cylindrical cyclone with a diameter of 0.20 m and a height of 1 m, the spray nozzle had an orifice with a diameter of 300 μm and was placed in the upper part of the recipient with a tangential orientation inducing a movement of rotation on the gaseous flow generated in the annular space included between the outer wall of the cyclone and the inner cylinder 12 having a diameter of 0.16 m and a height of 0.40 m.

### EXAMPLE 1

In the equipment thus described, very fine particles of L-ascorbic acid, which were obtained by an anti-solvent method and whose mean diameter by mass is 2.3 μm, are encapsulated in a lipidic texture agent conventionally used in the food industry, constituted by hydrogenated vegetable oil, and marketed by the Société Industrielle des Oléagineux under code GV 60 whose melting occurs at atmospheric pressure between 58° and 61° C.

The suspension is made at atmospheric pressure at 62° C. by stirring 2 kg of a mixture comprising 1.800 kg of GV 60 and 0.200 kg of L-ascorbic acid for 15 mintues in the enclosure 1 of which the double envelope has water passing therethrough at 60° C. and the stirrer is rotated at 200 revs per minute. Carbon dioxide is then introduced at 60° C. until the pressure is stabilized at 6 MPa. After 20 minutes' stirring, in order to produce a perfect equilibrium, the enclosure 1 is placed in communication with the nozzle 9 and the heavy phase present in the enclosure 1 is sprayed into the collector recipient 11. During this phase, the pressure is maintained by the admission of fluid into the enclosure and the stirring is likewise maintained. After 10 minutes, 0.320 kg of white powder is collected, of which the analysis of the characteristics leads to the following results:

Granulometric distribution included between 5 μm and 19 μm with a mean diameter by mass of 12 μm;

Mean composition of the particles, obtained after dissolution of the coating agent in heptane and of the vitamin in water, by HPLC over silica column C18 with UV detection (254 nm): 9.5% mass of L-ascorbic acid.

### EXAMPLE 2

The test made in Example 1 is reproduced under slightly different conditions. This time, the temperature in the enclosure 1 is maintained at 50° C. only, with the result that this temperature is insufficient to obtain a liquid phase even in the presence of the fluid at 6 MPa. Prior to its introduction, 0.360 kg of pure ethanol was therefore added to the coating agent and 2.160 kg of this mixture was introduced in the enclosure 1. Furthermore, all the other parameters were maintained equal to those used in Example 1, and 0.338 kg of powder was obtained, of which the analysis of the characteristics led to results very close to those obtained on the powder described in Example 1:

Granulometric distribution included between 12 μm and 38 μm with a mean diameter by mass of 27 μm;

Mean composition of the particles obtained by HPLC: 9.7% by mass of L-ascorbic acid.

Moreover, the analysis by gaseous phase chromatography of the ethanol content of the particles comprising this powder leads to a content of 0.4% by mass.

Measurements relative to the placing of the L-ascorbic acid in solution in the water were conducted at 20° C. The curve presented in FIG. 2 represents, as a function of the time expressed in hours, the percentage by mass % m of acid which dissolved in the water with respect to the total quantity of acid present in the particles. It is observed that the acid was effectively encapsulated within the coating agent without immediate effect of salting out during the placing in contact with water. On the contrary, a very regular progressive salting out of the acid was observed for 36 hours.

For certain uses of the microcapsules thus obtained, it may be important that the percentage of remaining ethanol be very low. In that case, a “stripping” is effected, i.e. a treatment of extraction with the aid of a fluid at supercritical pressure particularly constituted by carbon dioxide. In this way, a sample constituted by 100 g of microcapsules of L-ascorbic acid obtained according to Example 2 was disposed in an autoclave of 0.5 litre within which a mass of 5 kg of pure carbon dioxide in supercritical state at a pressure of 10 MPa and at a temperature of 33° C. was made to percolate for an hour. Analysis by gaseous phase chromatography showed that the content of residual ethanol present in the microcapsules passed from 0.4% by mass to 0.02% by mass without the characterization of the particles which was effected after treatment showing any modification of their structure.

### EXAMPLE 3

The test made in Example 2 is reproduced under virtually identical conditions, except that the organic solvent added to the coating agent is acetone and not ethanol. Prior to its introduction, 0.360 kg of pure acetone was therefore added to the coating agent and 2.160 kg of this mixture were therefore introduced into the enclosure 1. Furthermore, all the other parameters were maintained equal to those used in Example 2, and 0.332 kg of powder was obtained of which the analysis of the characteristics led to results very close to those obtained for the powders described in Examples 1 and 2:

Granulometric distribution included between 11 μm and 29 μm with a mean diameter by mass of 21 μm;

Mean composition of the particles, obtained by HPLC: 9.8% by mass of L-ascorbic acid.

Analysis by gaseous phase chromatography of the acetone content of the particles composing this powder led to a content of 0.12% by mass, or 1200 ppm.

### EXAMPLE 4

The test made in Example 2 is reproduced under the same conditions by using another coating agent, namely a silicone wax of AMS-C30 type by Dow Coming, currently used as excipient in cosmetic products. The temperature in the enclosure 1 is maintained at 50° C., and the pressure at 6 MPa. Prior to its introduction, 0.600 kg of pure ethanol was added to the coating agent and 2.400 kg of this mixture were thus introduced into the enclosure 1. Furthermore, all the other parameters were maintained equal to those used in Example 2, and 0.319 kg of powder was obtained of which the analysis of the characteristics led to results very close to those obtained on the powder described in Example 1:

Granulometric distribution included between 25 μm and 42 μm with a mean diameter by mass of 32 μm;

Mean composition of the particles: 9.2% by mass of L-ascorbic acid.

Moreover, the analysis by gaseous phase chromatography of the ethanol content of the particles composing this powder leads to a content of 0.5% by mass.

### EXAMPLE 5

The test made in Example 1 is reproduced under virtually identical conditions except that the active agent is constituted by fine particles of albumin obtained from egg also called ovalbumin, with a granulometry centred on 5 μm. 1.800 kg of coating agent GV 60 and 0.180 kg of albumin were therefore introduced in the enclosure 1. Furthermore, all the other parameters were maintained equal to those used in Example 1, in particular the temperature was maintained at 60° C. and the pressure at 6 MPa in the enclosure. 0.310 kg of powder was obtained of which the analysis of the characteristics led to results very close to those obtained on the powder described in Example 1:

Granulometric distribution included between 4 μm and 22 μm with a mean diameter by mass of 10 μm;

Mean composition of the particles obtained after dissolution of the excipient in heptane and of the protein in isotonic solution by UV spectrophotometry (280 nm): 10.2% by mass of albumin.

Measurements relative to the placing of the albumin in solution in the isotonic solution were likewise conducted at 37° C. with following of the concentration in the water by UV spectrophotometry. The curve of salting out of the albumin as a function of time is of the same type as that presented in FIG. 2, showing that the albumin was effectively encapsulated within the coating agent without immediate effect of salting out during the placing in contact with the aqueous phase. On the contrary, a very regular progressive salting out of the albumin was observed for 48 hours.

### EXAMPLE 6

The test made in Example 5 was reproduced under virtually identical conditions, except that the active agent is constituted by fine particles of a protein called lactase with a granulometry centred on 6 μm. 1.800 kg of coating agent GV60 and 0.180 kg of lactase were therefore introduced into the enclosure 1. Furthermore, all the other parameters were maintained equal to those of Example 5 in particular the temperature was maintained at 60° C. and the pressure at 6 MPa. 0.305 kg of powder was obtained of which the analysis of the characteristics led to results very close to those obtained on the powder described in Example 1:

Granulometric distribution included between 10 μm and 24 μm with a mean diameter by mass of 18 μm;

Mean composition of the particles obtained after dissolution of the excipient in heptane and of the protein in isotonic solution by UV spectrophotometry (280 nm): 9.4% by mass of protein.

Measurements relative to the biological activity of the protein were conducted in accordance with the protocol generally used for measuring the enzymatic activity of lactases: The reaction employed is the hydrolysis of O-nitrophenyl-galactopyranoside(ONPG) in O-nitrophenol and D-galactose, the production of O-nitrophenol being followed by spectrophotometry at 420 nm. The activity of the starting lactase was found equal to 542000 units/gram (±15 000) and the activity of the lactase after encapsulation to 454000 units/grams (±28 000), viz. a loss of activity of about 16%.

Measurements relative to the placing of the protein in solution in the isotonic solution were likewise conducted at 37° C. with following of the concentration in the water by UV spectrophotometry. The curve of salting out of the protein as a function of time is of the same type as that shown in FIG. 2, showing that the protein was effectively encapsulated within the coating agent without immediate effect of salting out during the placing in contact with the aqueous phase. On the contrary, a very regular progressive salting out of the protein was observed for 8 hours.

### EXAMPLE 7

The test made in Example 6 is reproduced under virtually identical conditions with the same active agent and the same excipient, the temperature being maintained at 50° C. and the pressure at 6 MPa in the enclosure 1. However, as described in Example 2, 1.800 kg of coating agent GV60 and 0.420 kg of ethanol, as well as 0.180 kg of lactase were introduced at the same time in the enclosure 1. 0.320 kg of powder was obtained, of which the analysis of the characteristics led to results very close to those obtained on the powder described in Example 1:

Granulometric distribution included between 28 μm and 62 μm with a mean diameter by mass of 51 μm;

Mean composition of the particles obtained after dissolution of the excipient in heptane and of the protein in isotonic solution by UV spectrophotometry (280 nm): 9.5% by mass of protein.

Measurements relative to the biological activity of the protein were conducted in accordance with the protocol described in Example 6. The activity of the starting lactase was found equal to 590000 units/gram (±12000) and the activity of the lactase after encapsulation to 588000 units/gram (±15000), viz. a negligible loss of activity.

Moreover, the analysis by gaseous phase chromatography of the ethanol content of the particles composing this powder led to a content of 0.5% by mass.

A comparison of the results obtained in Examples 6 and 7 shows that the implementation of the method at subcritical pressure, on the one hand does not bring about considerable denaturation of the protein, unlike what is stated in the prior art when carbon dioxide at supercritical pressure is used, and, on the other hand that the ethanol does not contribute to the denaturation of the protein.

1. Method for encapsulating fine solid particles in the form of microcapsules, characterized in that it comprises the steps consisting in:
forming, in an enclosure (1), a suspension of these particles in a liquid constituted by at least one coating agent in intimate contact with a fluid at a pressure lower than its critical pressure so as to provoke saturation of said suspension with this fluid,
spraying the fluid-saturated suspension through expansion means, and
collecting the microcapsules in the gas stream resulting from decompression.

2. Method of encapsulation according to claim 1, characterized in that, during the first step, an aqueous or organic solvent is added to the coating agent.

3. Method according to claim 2, characterized in that the solvent is entrained by the gaseous fluid.

4. Method according to one of claims 2 or 3, characterized in that a “stripping” of the microcapsules is effected by a fluid taken to a supercritical pressure.

5. Method according to one of the preceding claims, characterized in that the fluid at subcritical pressure is constituted by carbon dioxide.

6. Method according to one of the preceding claims, characterized in that a continuous supply is ensured of the fluid at subcritical pressure and of the suspension of the active agent in the coating agent inside an enclosure (1) as well as a continuous drawing off of the products contained in this enclosure which are admitted into a spray nozzle (9) disposed in a collector recipient (11) maintained at a pressure close to atmospheric pressure.

7. Method according to any one of the preceding claims, characterized in that a lipid or a mixture of lipids is used as coating agent.

8. Method according to one of the preceding claims, characterized in that the placing in suspension of the particles is effected at a temperature of the order of 50° C. to 60° C. and at a pressure of the order of 5 Mpa to 6 Mpa and the pressure during the collection is close to atmospheric pressure.

9. Microcapsules obtained in accordance with the method object of one of claims 1 to 8, characterized in that the particles are constituted by an active agent of alimentary, pharmaceutical, cosmetic, agrochemical or veterinary interest.

10. Microcapsules obtained according to claim 9, characterized in that the particles are constituted by a protein.

11. Microcapsules according to claim 10, characterized in that the proteins are associated with a stabilization agent.
